(19) United States
(12) Patent Application Publication
(10) Pub. No.: US 2008/0228123 A1
(43) Pub. Date: Sep. 18, 2008

(54) BANDAGE WITH A HYDROPHILIC FOAM CONTAINING SILVER AND A HEMASTATIC AGENT

(75) Inventors: Michael Moore, Clarks Summit, PA (US); Jeffrey Glattly, Dalton, PA (US); William F. McNally, Clarks Summit, PA (US)

Correspondence Address: AKERMAN SENTERFITT, P.O. BOX 3188, WEST PALM BEACH, FL, 33402-3188, US

(73) Assignee: Noble Fiber Technologies, LLC, Scranton, PA (US)

(21) Appl. No.: 12/035,164

(22) Filed: Feb. 21, 2008

Related U.S. Application Data
(60) Provisional application No. 60/894,777, filed on Mar. 14, 2007.

(51) Int. Cl. A61F 13/00; A61L 15/08

(52) U.S. Cl. 602/46; 602/48; 602/53

(57) ABSTRACT

A compression stretch bandage formed from a hydrophillic foam having at least one layer of a stretchable, textile material attached to one side of the foam and a hemastasis agent attached to another side of the foam for activating the intrinsic arm of the hemostatic cascade of the healing process. The bandage may also include a silver material contained within the foam for reducing risk of infection. The bandage may be a flexible, stretchable, hydrophilic bandage that reduces the risk of infection at a wound by providing a moist environment that will aid in optimum release of silver ions into the wound.

### CROSS REFERENCE TO RELATED APPLICATION

This application claims the benefit of U.S. Provisional Application No. 60/894,777, filed Mar. 14, 2007.

### FIELD OF THE INVENTION

This invention is directed generally to an antibiotic bandages, and more particularly to wound dressing bandages that foster a moist wound-healing environment while minimizing the possibility of infection and facilitating hemostasis.

### BACKGROUND

Silver has been used as an antimicrobial since ancient times. Recent years have seen a renewed interest in silver as an antibiotic. This renewed interest is driven in part by the development of antibiotic-resistant bacteria, such as methicillin-resistant Staphylococcus aureus (MRSA). Resistant bacteria are especially problematic in wounds. Silver is a broad-spectrum antibiotic that is effective against such resistant bacteria and bacteria do not appear to develop resistance to silver. There is an urgent need for an antibiotic wound care product that uses silver to treat and/or prevent MRSA and other resistant infections.

Silver is also known to exhibit wound-healing properties. Expeditious wound healing benefits the patient in terms of increased comfort and decreased susceptibility to infection and secondary injury. There is a need for wound care products that utilize silver to increase the rate of wound healing.

Many presently existing antibiotic wound care products lose their antibiotic activity in a short period of time. Such dressings must be replaced frequently often resulting in extreme pain or discomfort and inconvenience for the patient as the dressing is removed and a new dressing is applied. Similarly, silver creams (including silver sulfadiazine) must be consistently reapplied to the injured area, and the dressing must be removed for reapplication of the cream. There is a need for a wound care product that releases silver ions over an extended period of time and which alleviates the need for frequent removal or replacement of the dressing or application of silver creams.

Silver is commonly applied in as a silver salt. Such salts can be irritating to the skin. There is a need for a non-irritating silver wound care product that does not rely on silver salts for the delivery of silver ions. Moreover, prolonged contact with silver salts can cause argyria, which creates a pronounced, permanent ashen-gray skin discoloration that can be localized or universal. Thus, there is a need for a silver wound care product that does not cause argyria.

Silver is known to affect the operation of matrix metalloproteinases (MMPs). Excessive MMPs are known to interfere with wound healing. Excessive interference with MMPs can also interfere with wound healing. Presently existing silver-based wound care products often inhibit MMPs too much, thereby interfering with the wound healing process. Thus, there is a need for a silver wound care product that delivers an amount of silver, which limits the activity of MMPs without unduly restricting MMP activity.

### SUMMARY OF THE INVENTION

This invention is directed to a bandage formed from a hydrophillic foam having a hemastasis agent attached to the foam for activating the intrinsic arm of the hemastatic cascade of the healing process. The hemastasis agent may be applied to the bandage in many different manners, such as, but not limited to, throughout the foam, on both sides of the foam or attached to one side of the bandage. The bandage may also include a silver material contained within the foam for reducing risk of infection. In one embodiment, the bandage may include at least one layer of a stretchable, textile material attached to one side of the foam opposite to the hemastasis agent that forms a compression stretch bandage. The bandage may be a flexible, stretchable, hydrophilic bandage that reduces the risk of infection at a wound by providing a moist environment that aids in optimum release of silver ions into the wound.

The bandage may be formed from a foam material having a plurality of pores therethrough, wherein the foam material forms the body of the bandage. IN one embodiment, the bandage may also include at least one layer of a stretchable, textile material attached to an outer surface of the foam material on a first side of the foam material, a silver material attached to the foam material for reducing risk of infection, and a hemastasis agent attached to an outer surface of the foam material on a second side that is generally opposite to the first side. The foam material may be formed from polyurethane, a mesoporous bioactive glass bead, a hydrophilic foam, a compressible foam, or other appropriate material. The hydrophilic foam may be formed from polyurethane or other materials with porous foam channels that allow the transfer of fluids through the foam. In one embodiment, the at least one layer of a stretchable, textile material may be comprised of an elastic compression wrap. The silver material may be a silver hydroxide, a silver powder or other appropriate material. The silver material may be included in the foam material such that the silver material is positioned through the base material. In one embodiment, the silver material may be silver coated fibers, such as silver coated nylon fibers.

An advantage of this invention is that the bandage may include a specialized metallic silver powder and silver hydroxide to provide ionic silver to treat and/or prevent MRSA and other resistant bacteria and fungi.

Another advantage of this invention is that the bandage uses ionic silver to inhibit the growth of bacteria that is detrimental to wound healing.

Yet another advantage of this invention is that the bandage creates and maintains a moist wound-healing environment while preventing the growth of bacteria and fungi.

Still another advantage of this invention is that the bandage includes a hemastasis agent for accelerating the clotting process.

Another advantage of this invention is that the bandage is a compression bandage thereby enabling the bandage to be used to apply continuous pressure to a wound.

Yet another advantage of this invention is that the bandage maintains a moist environment, but eliminates unpleasant odors.

Another advantage of this invention is that the bandage uses metallic silver with a relatively large surface area that does not become detached from the base material.

Still another advantage of this invention is that the bandage enables the delivery of a dosage of silver ions.

Another advantage of this invention is that the bandage delivers an amount of silver that limits the activity of MMPs without unduly restricting MMP activity.

Yet another advantage of this invention is that the bandage is easy and inexpensive to manufacture.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a cross-sectional perspective view of a bandage having aspects of this invention.

FIG. 2 is a cross-sectional perspective view of another embodiment of the bandage.

### DETAILED DESCRIPTION OF THE INVENTION

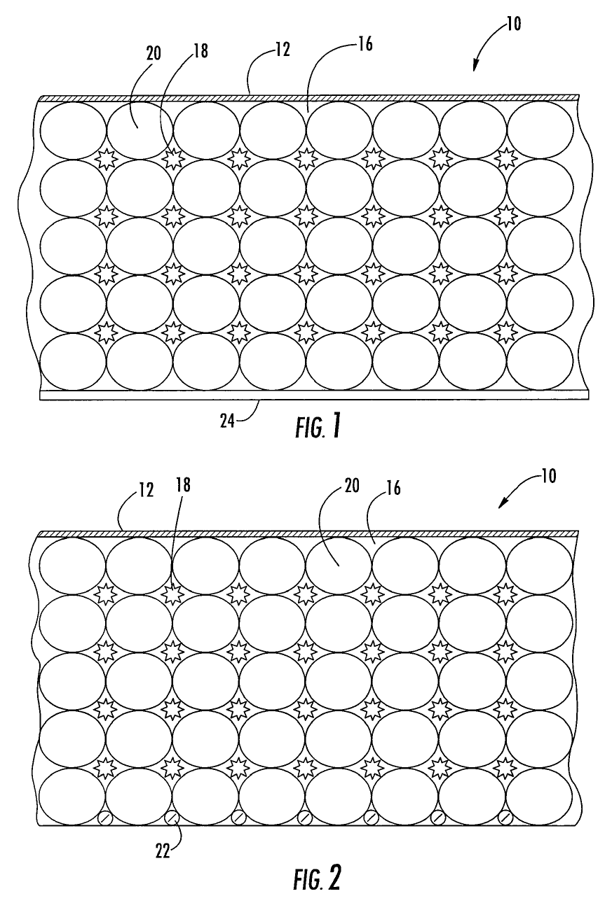

As shown in FIGS. 1 and 2, this invention is directed to a bandage 10 formed from a base material 16, which may be a hydrophilic foam, having a hemastasis agent attached to a side of the foam 16 for activating the intrinsic arm of the hemastatic cascade of the healing process. A silver material 18 may be attached to the hydrophilic foam for reducing risk of infection. The bandage 10, and specifically, the hydrophilic foam, may create a moist environment that is optimum for the release of silver ions into the wound bed to reduce the risk of infection in a wound on a patient. In one embodiment, a stretchable, textile material 12 may be attached to a first side of the foam opposite to the hemastasis agent.

The foam material 16 may be formed from a hydrophilic foam or other appropriate foam material. The hydrophilic foam 16 may be formed from a honeycomb structure, as shown in FIGS. 1 and 2, in which a plurality of channels 20 extend throughout the hydrophilic foam material 16. The channels 20 may extend for any length within the foam 16. The hydrophilic foam 16 may be formed from a compressible foam with open passages, such as channels 20, that allow fluid to migrate into the foam 16 as the absorptive capacity of the foam 16 becomes saturated during use. This action enhances the diffusion gradient between the inner portion of the bandage 10 and the outer portion of the bandage 10. The hydrophilic foam 16 can be constructed from many substances, such as, but not limited to, polyurethane and glass. If the foam 16 is made from glass beads, such as mesoporous bioactive glass beads, the glass includes a porous component.

In one embodiment, as shown in FIGS. 1 and 2, the bandage 10 may include a textile material 12. The textile material 12 may be a compression stretch bandage. The textile material 12 may be any appropriate material. The textile material 12 may be formed into a roll, such as between about ½ inch and 5 inches wide and between about 2 inches and 3 feet in length. The textile material 12 may be attached to the foam material 16 in any appropriate method, such as through the use of adhesives, stitching and other appropriate methods.

The foam 16 may be metallized with silver 18 for antimicrobial purposes. The silver material 18 may be attached to an outer surface of the base material 16, referred to as metallizing the base material 16, or may be incorporated in the base material 16 during formation of the base material 16, as shown in FIG. 4. The silver material 18 may be attached to the base material 16 via metallizing whereby the silver material 18 forms a layer of silver on the base material 16. In another embodiment, the silver material 18 may be included in the base material 16 during formation of the base material 16. By including the silver material 18 in the base material 16, the silver material 18 may found throughout the base material 16 and not limited to being only on the surface of the base material 16. The silver 18 may be formed on the foam 16 in any appropriate way.

In one embodiment, the silver material 18 may be formed from a silver hydroxide or other appropriate material. The silver hydroxide may be prepared as described below. In another embodiment, the silver material 18 may be a silver micro and nano powder. The silver micro and nano powder may also be prepared as described below. The silver powder may be surface modified if a need for a greater ion release exists for a particular application.

The silver 18 may be obtained according to the following method. The first step is to take a source of the metal, such as silver nitrate powder for embodiments wherein silver particles are to be formed, and dissolve it in water. In one embodiment, the water is de-ionized water. In an alternative embodiment, a pre-dissolved silver nitrate solution may be used provided the amount of water in the solution is known. The table below references to different embodiments for various amounts of silver nitrate and water that may be used in an embodiment wherein the source of silver is silver nitrate and de-ionized water is used.

| | Amount of Silver Nitrate in |
|---|---|
| | gm per liter of DI water |
| First | about 3 to about 500 |
| Embodiment | |
| Second | about 50 to about 350 |
| Embodiment | |
| Third | about 100 to about 200 |
| Embodiment | |
| Fourth | about 150 |
| Embodiment | |

The above mentioned solution may then treated with an alkali solution. In one embodiment, the alkali solution is sodium hydroxide. Sodium hydroxide may be used due to its great tendency to complex with the metallic solution. However, any alkali solution that is able to complex with the metallic solution used in a particular embodiment may be used in the present invention. In this embodiment, the metallic solution is silver nitrate dissolved in DI water. Other alkali solutions that may be used include, but are not limited to, ammonium hydroxide.

The table below references to different embodiments of the present invention for various amounts of sodium hydroxide that may be used in those embodiments wherein sodium hydroxide is the alkali solution. It should be noted that the starting point to make the solution is 50% sodium hydroxide solution (50:50 v/v) which is readily available from multiple vendors.

| | ml of NaOH from |
|---|---|
| | 50:50 |
| First | about 10 to about |
| Embodiment | 500 |
| Second | about 50 to about |
| Embodiment | 300 |
| Third | about 75 to about |
| Embodiment | 150 |
| Fourth | about 100 |
| Embodiment | |

The reaction may take place at room temperature, or at a temperature of from about 15 to about 30° C. During the reaction, the alkali solution complexes with the metal to form a precipitate containing the metal. In those embodiments wherein sodium hydroxide is used, brown precipitate is formed as the sodium hydroxide is added. The solution may be stirred while the precipitate is forming. After all the alkali solution is added, the resultant mixture may be allowed to settle down for a period of time to permit settling of any precipitate. The amount of time permitted for settling may vary, but may be from about 5 to about 15 minutes.

After settling, the precipitate is then removed. The precipitate may be filtered using standard filter paper, such as a Buckner funnel. Depending on the pH of the solution, the solution may be neutralized. As the alkali solution will generally increase the pH to above 7, an acid may be used to bring the solution to a pH of approximately 7. In one embodiment, sulfuric acid may be used, although other acids may also be used including, but not limited to, hydrochloric acid and nitric acid, among others. Bringing the pH of the solution to about 7 is beneficial because such pH will facilitate easy processing from waste treatment point of view, although this step is not necessary in the formation of the micro-sized and nano-sized particles of the present invention.

The precipitate is then rinsed with water, such as deionized water. The water is, beneficially, used to wash the precipitate thoroughly. Washing of the precipitate helps facilitate nano- and micro-sized particles of the complexed metal precipitate to be collected in pure form. In an embodiment wherein silver is the metal and sodium hydroxide is the alkali solution, the resulting precipitate includes nano- and micro-sized particles of silver hydroxide. The rinsing may be done anywhere, including within the funnel itself.

The precipitate may then be dried in a conventional oven or other drying mechanism until the precipitate is substantially dry. In one embodiment, the drying temperature is from about 50 to about 90° C. After drying the precipitate, the resulting product includes the micro-sized and/or nano-sized particles of the present invention. The silver 18 may provide one or more of these properties including, but are not limited to, anti-microbial, anti-fungal, anti-static, conductive, electromagnetic interference (EMI) shielding, filtration, or a combination thereof.

An outer surface of the foam 16 may be covered with a hemastasis material 24 that may accelerate the clotting process. In another embodiment, the hemastasis agent 24 is not attached to the foam 16. The hemastasis agent may be formed from collagen, cellulose, polysaccharides, mesoporous glass, gelatin, thrombin, fibrinogen, aluminum sulfate, zeolyte, epsilon aminocaproic acid, crustacean protein, or other appropriate materials.

During use, the foam 16 absorbs exudates from the wound. The exudates are contained in the channels 20 of the hydrophilic foam 16 and exposed to the silver 18 within the hydrophilic foam 16 due to the open cell construction. The foam 16 design acts as a matrix to activate the extrinsic clotting mechanism and facilitates clotting within the foam. The topical hemastasis agent 24 activates the intrinsic clotting pathway and is in direct contact with the open wound. In another embodiment, the hemastasis agent 24 could also be within the foam as well.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A bandage, comprising:
a foam material having a plurality of pores therethrough, wherein the foam material forms a body of the bandage;
a silver material attached to the foam material for reducing risk of infection; and
a hemastasis agent attached to an outer surface of the foam material on a second side that is generally opposite to the first side.

2. The bandage of claim 1, further comprising at least one layer of a stretchable, textile material attached to an outer surface of the foam material on a first side of the foam material.

3. The bandage of claim 2, wherein the at least one layer of a stretchable, textile material is comprised of an elastic compression wrap.

4. The bandage of claim 1, wherein the foam material may be formed from a mesoporous bioactive glass bead.

5. The bandage of claim 1, wherein the foam material comprises a hydrophilic foam.

6. The bandage of claim 5, wherein the hydrophilic foam is formed from polyurethane with porous foam channels that allow the transfer of fluids through the foam.

7. The bandage of claim 1, wherein the silver material comprises a silver hydroxide.

8. The bandage of claim 1, wherein the silver material comprises a silver powder.

9. The bandage of claim 1, wherein the silver material is included in the foam material such that the silver material is positioned throughout the base material.

10. A bandage, comprising:
a hydrophilic foam material formed from a honeycomb structure including a plurality of channels therethrough, wherein the foam material forms a body of the bandage;
a silver material attached to the foam material for reducing risk of infection; and
a hemastasis agent attached to an outer surface of the foam material on a second side that is generally opposite to the first side.

11. The bandage of claim 10, further comprising at least one layer of a stretchable, textile material attached to an outer surface of the foam material on a first side of the foam material.

12. The bandage of claim 11, wherein the at least one layer of a stretchable, textile material is comprised of an elastic compression wrap.

13. The bandage of claim 10, wherein the foam material may be formed from a mesoporous bioactive glass bead.

14. The bandage of claim 10, wherein the foam material comprises a compressible foam.

15. The bandage of claim 10, wherein the hydrophilic foam is formed from polyurethane with porous foam channels that allow the transfer of fluids through the foam.

16. The bandage of claim 10, wherein the silver material comprises a silver hydroxide.

17. The bandage of claim 10, wherein the silver material comprises a silver powder.

18. The bandage of claim 10, wherein the silver material is included in the foam material such that the silver material is positioned throughout the base material.
